SEQUENCE LISTING (9 sequences)

SEQ ID NO: 1 - protein, 277 aa, Homo sapiens
  helicase ATP-binding domain of ERCC2 (1)..(277)
GLLVYFPYDYIYPEQFSYMRELKRTLDAKGHGVLEMPSGTGKTVSLLALIMAYQRAYPLEVTKLIYCSRTVPEIEKVIEELRKLLNFYEKQEGEKLPFLGLALSSRKNLCIHPEVTPLRFGKDVDGKCHSLTASYVRAQYQHDTSLPHCRFYEEFDAHGREVPLPAGIYNLDDLKALGRRQGWCPYFLARYSILHANVVVYSYHYLLDPKIADLVSKELARKAVVVFDEAHNIDNVCIDSMSVNLTRRTLDRCQGNLETLQKTVLRIKETDEQRLRD

SEQ ID NO: 2 - protein, 760 aa, Homo sapiens
  human ERCC2 protein sequence (1)..(760)
MKLNVDGLLVYFPYDYIYPEQFSYMRELKRTLDAKGHGVLEMPSGTGKTVSLLALIMAYQRAYPLEVTKLIYCSRTVPEIEKVIEELRKLLNFYEKQEGEKLPFLGLALSSRKNLCIHPEVTPLRFGKDVDGKCHSLTASYVRAQYQHDTSLPHCRFYEEFDAHGREVPLPAGIYNLDDLKALGRRQGWCPYFLARYSILHANVVVYSYHYLLDPKIADLVSKELARKAVVVFDEAHNIDNVCIDSMSVNLTRRTLDRCQGNLETLQKTVLRIKETDEQRLRDEYRRLVEGLREASAARETDAHLANPVLPDEVLQEAVPGSIRTAEHFLGFLRRLLEYVKWRLRVQHVVQESPPAFLSGLAQRVCIQRKPLRFCAERLRSLLHTLEITDLADFSPLTLLANFATLVSTYAKGFTIIIEPFDDRTPTIANPILHFSCMDASLAIKPVFERFQSVIITSGTLSPLDIYPKILDFHPVTMATFTMTLARVCLCPMIIGRGNDQVAISSKFETREDIAVIRNYGNLLLEMSAVVPDGIVAFFTSYQYMESTVASWYEQGILENIQRNKLLFIETQDGAETSVALEKYQEACENGRGAILLSVARGKVSEGIDFVHHYGRAVIMFGVPYVYTQSRILKARLEYLRDQFQIRENDFLTFDAMRHAAQCVGRAIRGKTDYGLMVFADKRFARGDKRGKLPRWIQEHLTDANLNLTVDEGVQVAKYFLRQMAQPFHREDQLGLSLLSLEQLESEETLKRIEQIAQQL

SEQ ID NO: 3 - protein, 17 aa, Homo sapiens
  Motif I, amino acid positions 35-51 (1)..(17)
KGHGVLEMPSGTGKTVS

SEQ ID NO: 4 - protein, 20 aa, Homo sapiens
  Motif IA, amino acid positions 69-88 (1)..(20)
KLIYCSRTVPEIEKVIEELR

SEQ ID NO: 5 - protein, 15 aa, Homo sapiens
  Motif II, amino acid positions 225-239 (1)..(15)
LARKAVVVFDEAHNI

SEQ ID NO: 6 - protein, 14 aa, Homo sapiens
  Motif III, amino acid positions 455-468 (1)..(14)
IITSGTLSPLDIYP

SEQ ID NO: 7 - protein, 22 aa, Homo sapiens
  Motif IV, amino acid positions 533-554 (1)..(22)
DGIVAFFTSYQYMESTVASWYE

SEQ ID NO: 8 - protein, 27 aa, Homo sapiens
  Motif V, amino acid positions 587-613 (1)..(27)
ACENGRGAILLSVARGKVSEGIDFVHH

SEQ ID NO: 9 - protein, 18 aa, Homo sapiens
  Motif V, amino acids 654-671 (1)..(18)
FDAMRHAAQCVGRAIRGK

(19) United States
(12) Patent Application Publication
(10) Pub. No.: US 2017/0081724 A1
(43) Pub. Date: Mar. 23, 2017

(54) METHOD AND KITS FOR DETERMINING SENSITIVITY TO CANCER TREATMENT

(71) Applicants: Dana-Farber Cancer Institute, Inc., Boston, MA (US); Memorial Sloan-Kettering Cancer Center, New York, NY (US)

(72) Inventors: Jonathan Eric Rosenberg, New York, NY (US); Eliezer Mendel Van Allen, Brookline, MA (US); Levi Alexander Garraway, Newton, MA (US); Philip Wayne Kantoff, Brookline, MA (US); Nikhil Wagle, Boston, MA (US)

(21) Appl. No.: 15/126,423

(22) PCT Filed: Mar. 17, 2015
(86) PCT No.: PCT/US15/21014

§ 371 Date: Sep. 15, 2016

Related U.S. Application Data
(60) Provisional application No. 61/955,432, filed on Mar. 19, 2014.

(51) Int. Cl. C12Q 1/68; A61K 33/24

(52) U.S. Cl. CPC C12Q 1/6886; A61K 33/24; C12Q 2600/106; C12Q 2600/156

(57) ABSTRACT

A method of increasing the efficacy of treatment for a patient suffering from urothelial carcinoma. The method comprises the steps of determining the presence or absence in a biological sample from the patient of somatic ERCC2 mutation followed by performing appropriate treatment. The absence of somatic ERCC2 mutation indicates that the urothelial carcinoma is likely to be unresponsive to cisplatin chemotherapy, and the patient then undergoes surgery to remove the carcinoma without accompanying cisplatin chemotherapy. The presence of somatic ERCC2 mutation indicates that the uroethelial carcinoma is likely to be responsive to cisplatin chemotherapy and the patient then undergoes surgery to remove the carcinoma accompanied by cisplatin chemotherapy.

This application claims the priority of U.S. Provisional Application Ser. No. 61/955,432, filed on Mar. 19, 2014.

### FIELD

The present application generally relates to methods of treatment for cancer using cisplatin-based chemotherapies and the detection of biomarkers relating to drug-resistant tumors.

### BACKGROUND

Platinum-based chemotherapy has been the standard of care for patients with muscle invasive and metastatic urothelial carcinoma for over 20 years. Neoadjuvant cisplatin-based combination chemotherapy leads to a 14-25% relative risk reduction for death from muscle invasive urothelial carcinoma. Pathologic downstaging from cT2-T4aN0M0 to pT0 or pTis at cystectomy occurs in 26-38% of patients treated with neoadjuvant chemotherapy compared to 12.3-15% for patients undergoing cystectomy alone. The 5-year survival for pT0/pTis patients is 85%, while only 43% of patients with persistent muscle invasive disease (≧T2) survive for 5 years after neoadjuvant chemotherapy. However, the inability to predict which patients will derive clinical benefit from neoadjuvant therapy has limited the use of this relatively toxic approach in the urological community.

### SUMMARY

One aspect of the present application relates to a method of determining sensitivity to cancer treatment in a patient suffering from cancer. In some embodiments, the method comprises the steps of: determining the presence of somatic mutation in one or more nucleotide excision repair genes in a biological sample from the patient, wherein the presence of one or more mutations in the one or more nucleotide excision repair genes indicates a sensitivity to the treatment by a platinum-based antineoplastic agent, wherein the cancer is selected from the group consisting of bladder cancer, gastric cancer, prostate cancer, colorectal cancer, lung adenocarcinoma, cutaneous melanoma, head and neck squamous cell carcinoma, low-grade glioma, cervical cancer, ovarian cancer, renal cancer and breast cancer, and wherein the one or more nucleotide excision repair genes comprise one or more genes elected from the group consisting of ERCC2, ERCC3 and ERCC5 genes.

Another aspect of the present application relates to method of treating a patient suffering from cancer. In some embodiments, the method comprises the steps of: determining the presence of somatic mutation in one or more nucleotide excision repair genes in a biological sample from the patient; and administering to the patient an effective amount of a platinum-based antineoplastic agent, if one or more mutations are found in the one or more nucleotide excision repair genes in the biological sample, wherein the cancer is selected from the group consisting of bladder cancer, gastric cancer, prostate cancer, colorectal cancer, lung adenocarcinoma, cutaneous melanoma, head and neck squamous cell carcinoma, low-grade glioma, cervical cancer, ovarian cancer, renal cancer and breast cancer, and wherein the one or more nucleotide excision repair genes comprise one or more genes elected from the group consisting of ERCC2, ERCC3 and ERCC5 genes.

Another aspect of the present application relates to a kit for determining sensitivity to a platinum-based antineoplastic agent in a cancer patient. In some embodiments, the kit comprises one or more synthetic oligonucleotides that specifically hybridizes to human ERCC2 gene; and one or more reagents for processing a biological sample to obtain nucleotide molecules.

### BRIEF DESCRIPTION OF DRAWINGS

FIG. 1 shows study design, mutation rates, and aggregate significant somatic mutations. FIG. 1A shows patients with muscle-invasive urothelial carcinoma cancer split into cases and controls based on their pathologic response to cisplatin-based neoadjuvant chemotherapy. Nine cases could not complete sequencing due to technical reasons (failed sequencing or elevated contamination). Tumors in FIG. 1B are arranged so that each column represents a tumor and each row represents a gene. The center panel is divided into responders (left and dark hashmarks) and non-responders (right and light hashmarks). The mutation rates of responders are elevated compared to non-responders in FIG. 1B (top of panel). The alteration landscape (center of FIG. 1B) of the aggregate cohort (n=50 patients) demonstrates a set of statistically significant genes that are altered in urothelial carcinoma (TP53, RB1, KDM6A, ARID1A). The negative log of the q values for the significance level of mutated genes is shown (for all genes with q<0.1) on the right side of FIG. 1B. ERCC2 mutation status is also shown below the other genes, although ERCC2 was not significantly mutated across the combined cohort. Additional data regarding allelic fraction ranges for each case (bottom of FIG. 1B), mutation rates (top of FIG. 1B), and mutational frequency (left of FIG. 1B) are also summarized in this figure.

FIG. 2 shows three tests examining selective enrichment of ERCC2 mutations in cisplatin-responder tumors. FIG. 2A shows a plot of MutSigCV gene-level significance (−log₁₀(MutSigCV p-value) and responder enrichment significance (−log₁₀ (Fisher's exact test p-value)). The size of the point is proportional to the number of responder patients who harbor alterations in the gene. Genes with a responder enrichment p-value of <0.01 are colored with dark hashmarks; others are colored with light hashmarks, and the dashed line denotes a p value of 0.01. Only ERCC2 reaches statistical significance in the responder cohort (P<0.001; Fisher's exact test). In FIG. 2B, among genes with sufficient number of alterations for cohort comparisons (n=9), only ERCC2 somatic mutations occur exclusively in the cisplatin responders, which is significant when accounting for the elevated mutation rate in responders compared to non-responders (P<0.05, denoted by asterisk). Compared to an unselected TCGA and Chinese urothelial carcinoma cohorts, FIG. 2C shows that ERCC2 somatic mutations are significantly enriched in the responder cohort (P<0.01, denoted by asterisk).

FIG. 3 shows mutation rates by cohort. FIG. 3A shows that the mutation rate for responders was higher than non-responders (p<0.001). Likewise, FIG. 3B shows that the mutation rate for ERCC2 mutant tumors was higher than in ERCC2 wild-type (WT) tumors (p=0.01).

FIG. 4 shows ERCC2 mutation mapping and distribution across tumor types. FIG. 4A depicts a stick plot of ERCC2 showing the locations of somatic mutations in the responders compared to ERCC2 mutations observed in two separate unselected bladder cancer exome cohorts. The ERCC2 mutations cluster within or near conserved helicase motifs. FIG. 4B illustrates the somatic ERCC2 mutation frequency in multiple tumor types. In FIG. 4C, the structure of an archaebacterial ERCC2 (PDB code: 3CRV) with mutations identified in the responder cohort mapped to their equivalent position is illustrated. These locations are shown in the context of canonical germline ERCC2 mutations responsible for xeroderma pigmentosum D (XPD), xeroderma pigmentosum/Cockayne Syndrome (XP/CS), and trichothiodystrophy (TTD).

FIG. 5 shows ERCC2 mutants fail to rescue cisplatin sensitivity of ERCC2-deficient cells. FIG. 5A, immunoblot of ERCC2 expression in cell lines created by transfection of the ERCC2-deficient parent cell line (GM08207; Coriell Institute), with pLX304 (Addgene) encoding GFP (negative control), WT ERCC2, or a mutant ERCC2. The negative control ERCC2-deficient cell line (lane 1) expresses endogenous levels of inactive ERCC2 from the parent cell genome, whereas WT (lane 2) and mutant (lanes 3-7) ERCC2 cell lines show increased levels of ERCC2 expressed from the transfected gene. 13-Actin is shown as a loading control. FIG. 5B, cisplatin sensitivity profiles of cell lines expressing WT or mutant ERCC2. Expression of WT ERCC2 in an ERCC2-deficient background rescues cisplatin sensitivity, whereas expression of the ERCC2 mutants fails to rescue cisplatin sensitivity. FIG. 5C shows IC₅₀ calculated from the survival data for each cell line. The difference in IC₅₀ between the parent (ERCC2-deficient) cell line and the cell line expressing WT ERCC2 was statistically significant, as was the difference between the WT ERCC2 cell line and each of the mutant ERCC2 cell lines (P<0.0001; ANOVA). The difference between the ERCC2-deficient cell line and each of the mutant cell lines was not statistically significant.

FIG. 6 shows ERCC2 mutants fail to rescue UV sensitivity of ERCC2-deficient cells. FIG. 6A, a representative colony formation assay for the ERCC2-deficient cell line (top) as well as the ERCC2-deficient line transfected with WT ERCC2 (middle), or one of the ERCC2 mutants (D609G, bottom) following increased doses of UV irradiation. FIG. 6B, clonogenic survival data for negative control, WT ERCC2, and mutant ERCC2. WT ERCC2 rescues UV sensitivity of the ERCC2-deficient cell line, whereas the mutant ERCC2s fail to rescue UV sensitivity. FIG. 6C, UV IC₅₀ values for cell lines. The difference between the ERCC2-deficient cell line and the WT ERCC2 cell line was significant (P<0.0001; ANOVA), whereas the difference between the ERCC2-deficient cell line and each of the ERCC2-mutant cell lines was not statistically significant (NS).

FIG. 7 shows ERCC2 mutants fail to rescue genomic instability following cisplatin exposure. FIG. 7A shows representative mitotic spreads from an ERCC2-deficient cell line. FIGS. 7B and 7C show the same ERCC2-deficient cell line transfected with WT ERCC2 (FIG. 7B), or one of the ERCC2 mutants (V242F; FIG. 7C) following cisplatin exposure. FIG. 7D shows chromosomal aberration data from ERCC2-deficient, WT ERCC2, and mutant ERCC2 cell lines. Rates of chromosomal aberrations following cisplatin exposure were significantly lower in the WT ERCC2 cell line than in the ERCC2-deficient line or the cell lines expressing mutant ERCC2 (P=0.03; ANOVA).

### DETAILED DESCRIPTION

The following detailed description is presented to enable any person skilled in the art to use the present methods and kits. For purposes of explanation, specific nomenclature is set forth to provide a thorough understanding of the present methods and kits. However, it will be apparent to one skilled in the art that these specific details are not required to practice the use of the methods and kits. Descriptions of specific applications are provided only as representative examples. The present methods and kits are not intended to be limited to the embodiments shown, but are to be accorded the widest possible scope consistent with the principles and features disclosed herein.

Headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). The terms “a” and “an” herein do not denote a limitation of quantity, but rather denote the presence of at least one of the referenced items.

As used herein the term “cancer” refers to any of the various malignant neoplasms characterized by the proliferation of cells that have the capability to invade surrounding tissue and/or metastasize to new colonization sites, including but not limited to carcinomas, sarcomas, melanoma and germ cell tumors. Exemplary cancers include bladder cancer, brain cancer, breast cancer, ovarian cancer, cervix cancer, colon cancer, head and neck cancer, kidney cancer, lung cancer, mesothelioma, prostate cancer, stomach cancer and uterus cancer. In one embodiment, the cancer is urothelial carcinoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which arises from transformed cells of mesenchymal origin. Sarcomas are malignant tumors of the connective tissue and are generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The term “nucleotide excision repair” as used herein, refers to a DNA repair mechanism. DNA damage occurs constantly because of chemicals (i.e., intercalating agents), radiation and other mutagens. Three excision repair pathways exist to repair single stranded DNA damage: nucleotide excision repair (NER), base excision repair (BER), and DNA mismatch repair (MMR). While the BER pathway can recognize specific non-bulky lesions in DNA, it can correct only damaged bases that are removed by specific glycosylases. Similarly, the MMR pathway only targets mismatched Watson-Crick base pairs. NER is a particularly important excision mechanism that removes DNA damage induced by ultraviolet light (UV). UV DNA damage results in bulky DNA adducts—these adducts are mostly thymine dimers and 6,4-photoproducts. Recognition of the damage leads to removal of a short single-stranded DNA segment that contains the lesion. The undamaged single-stranded DNA remains and DNA polymerase uses it as a template to synthesize a short complementary sequence. Final ligation to complete NER and form a double stranded DNA is carried out by DNA ligase.

NER functions are performed by the proteins transcribed from NER genes. Examples of NER genes include, but are not limited to, ERCC1, ERCC2, ERCC3, ERCC4, ERCC5, ERCC6, ERCC8, CCNH, CDK7, CETN2, DDB1, DDB2, LIG1, MNAT1, MMS19, RAD23A, RAD23B, RPA1, RPA2, TFIIH, XAB2, XPA and XPC.

The term “platinum-based antineoplastic agent,” as used herein, refers to a family of platinum-containing chemotherapeutic agents that are capable of crosslinking DNA as monoadduct, interstrand crosslinks, intrastrand crosslinks or DNA protein crosslinks. Examples of platinum-based antineoplastic agents include, but are not limited to, cisplatin, carboplatin, oxaliplatin, satraplatin, picoplatin, nedaplatin, triplatin and lipoplatin.

The term “alkylating antineoplastic agent” as used herein, refers to a family of chemotherapeutic agents that attaches an alkyl group (CₙH₂ₙ₊₁) to DNA. Examples of alkylating antineoplastic agents include, but are not limited to, nitrogen mustards such as cyclophosphamide, mechlorethamine or mustine (HN₂), uramustine or uracil mustard, melphalan, chlorambucil, ifosfamide and bendamustine; nitrosoureas such as carmustine, lomustine and streptozocin; alkyl sulfonates such as busulfan; triazenes such as dacarbazine, mitozolomide and temozolomide; procarbazine and altretamine.

### Method for Determining Sensitivity to Treatment

One aspect of the present application relates to a method of determining sensitivity to cancer treatment in a patient suffering from cancer, the method comprising the steps of determining the presence in a biological sample from the patient of somatic mutation associated with a nucleotide excision repair gene, wherein the presence of one or more mutations associated with nucleotide excision repair indicates a sensitivity to the treatment by a platinum-based antineoplastic agent or an alkylating antineoplastic agent.

In some embodiments, the cancer is carcinoma, sarcoma, melanoma or germ cell tumor. In other embodiments, the cancer is carcinoma. In some embodiments, the cancer is selected from the group consisting of bladder cancer, gastric cancer, prostate cancer, colorectal cancer, lung adenocarcinoma, cutaneous melanoma, head and neck SCC, low-grade glioma, cervical cancer, ovarian cancer, renal cancer and breast cancer. In other embodiments, the cancer is bladder cancer. In other embodiments, the cancer is urothelial carcinoma. In yet other embodiments, the cancer is muscle-invasive urothelial carcinoma.

In some embodiments, the somatic mutation is associated with one or more NER genes selected from the group consisting of ERCC1, ERCC2, ERCC3, ERCC4, ERCC5, ERCC6, ERCC8, CCNH, CDK7, CETN2, DDB1, DDB2, LIG1, MNAT1, MMS19, RAD23A, RAD23B, RPA1, RPA2, TFIIH, XAB2, XPA and XPC genes.

In some embodiments, the somatic mutation is associated with the ERCC2, ERCC3 or ERCC5 gene. In some embodiments, the somatic mutation is associated with the ERCC2 gene. In some embodiments, the mutation occurs at highly conserved amino acid positions within the helicase domains of ERCC2.

In some embodiments, the method further comprises the step of determining the presence in the biological sample of somatic mutation associated with one or more additional genes selected from the group consisting of ATM, PARP1, ATRX, PMS1, BAP1, PMS2, BARD1, ERCC5, POLE, BLM, FANCA, RAD50; MLH1, RAD51, BRCA1, BRCA2, MRE11A, RAD51B, BRIP1, MSH2, RAD51C, CHEK1, MSH6, RAD51D, CHEK2, NBN, RAD52, FANCC, PALB2, BRIP1, FANCG, FANCD2, FANCF, FANCL, FANCI, FANCJ and FANCB genes.

In some embodiments, somatic mutation is associated with the ERCC2 gene and the cancer is selected from the group consisting of bladder cancer, gastric cancer, prostate cancer, colorectal cancer, lung adenocarcinoma, cutaneous melanoma, head and neck SCC, low-frade glioma, cervical cancer, ovarian cancer, renal cancer and breast cancer.

In some embodiments, the mutation occurs at highly conserved amino acid positions adjacent to the helicase ATP binding domains of ERCC2. As used herein, the terms “adjacent to the helicase ATP binding domains of ERCC2,” “near the helicase ATP binding domains of ERCC2,” “adjacent to the conserved helicase motif of ERCC2,” “near the conserved helicase motif of ERCC2” refer to an amino acid position that is separated from the helicase ATP binding domain or conserved helicase motif of ERCC2 by 0, 1, 2, 3, 4, 5, 6, 7, 8, 9 or 10 amino acid residues. In other embodiments, the mutation is an ERCC2 mutation selected from the group consisting of mutations at amino acid positions 7 to 283 of the ERCC2 amino acid sequence (SEQ ID NO:1), which is also referred to as the “helicase ATP binding domain” of ERCC2.

In another embodiment, the mutations may be located throughout the complete ERCC2 protein sequence (SEQ ID NO:2).

The term “helicase ATP binding domain of ERCC2,” as used herein, refers to the position and type of a functional domain (amino acid residue 7-283, SEQ ID NO:1) within the amino acid sequence of human ERCC2 (SEQ ID NO:2). One of ordinary skill will understand that natural variants occur within the amino acid sequence and that the amino acid sequence described in the present application is a representative amino acid sequence and is not limiting upon the subject matter of the present application.

The term “highly conserved amino acid positions” refers to amino acid positions within the amino acid sequence that have maintained the identical amino acid in the identical position across species. One of ordinary skill will understand that highly conserved amino acid positions are likely to be positions at which the amino acid plays an important role in the functioning of a protein.

The term “conserved helicase motifs” refers to group positions of amino acid sequence homology across species. Conserved helicase motifs of ERCC2 comprise the following positions in the human ERCC2 amino acid sequence: Motif I, amino acid positions 35-51 (SEQ ID NO:3); Motif IA, amino acid positions 69-88 (SEQ ID NO:4); Motif II, amino acid positions 225-239 (SEQ ID NO:5); Motif III, amino acid positions 455-468 (SEQ ID NO:6); Motif IV, amino acid positions 533-554 (SEQ ID NO:7); Motif V, amino acid positions 587-613 (SEQ ID NO:8) and 654-671 (SEQ ID NO:9).

The human ERCC2 mutations that may be utilized in the methods disclosed herein include, but are not limited to, Y14C, Y24C, M42V, S44L, Y72C, E86G, S209C, N238S, V242F, S246Y, R286W, P463S, P463L, T484A/M, E606G, G607A, D609G/E, 11659Y, G665A, G675S, and Q758E. In some embodiments, human ERCC2 mutations that may be utilized in the methods disclosed herein include V242F, P463L, E606G, D609G and G665A. In some embodiments, the human ERCC2 mutations are located within or near the helicase ATP-binding domain (SEQ ID NO: 1) of ERCC2. In some embodiments, the human ERCC2 mutations are located within or near one or more of the conserved helicase motifs I (SEQ ID NO:3), IA (SEQ ID NO:4), II (SEQ ID NO:5), III (SEQ ID NO:6), IV (SEQ ID NO:7) and V (SEQ ID NOS: 8 and 9).

Somatic mutations associated with nucleotide excision repair genes can be determined with methods well known in the art. All techniques that are presently known, or which may be subsequently discovered, for the evaluation of somatic nucleotide mutations are contemplated for use with the present application. Techniques for evaluating the presence of somatic mutations in biological samples include microarray analysis, differential display, PCR, RT-PCR, Q-RT-PCR, Northern blots, Western blots, and Southern blots. Techniques that are contemplated for use with the present invention to detect mutations via sequencing include: Maxam-Gilbert sequencing; chain-termination sequencing; shotgun sequencing; bridge PCR; massively parallel signature sequencing (MPSS); polony sequencing; 454 pyrosequencing; Illumina (Solexa) sequencing; SOLiD sequencing; ion torrent semiconductor sequencing; DNA nanoball sequencing; heliscope single molecule sequencing; single molecule real time (SMRT) sequencing; nanopore DNA sequencing; tunneling currents DNA sequencing; sequencing by hybridization; sequencing with mass spectrometry; microfluidic Sanger sequencing; microscopy-based techniques; RNAP sequencing; in vitro virus high-throughput sequencing; whole-genome sequencing (WGS), whole exome sequencing (WES); whole transcriptome shotgun sequencing (WTSS). Other methods, such as ultiplex ligation-dependent probe amplification (MLPA), single strand conformational polymorphism (SSCP), denaturing Gradient Gel Electrophoresis (DGGE), heteroduplex analysis and restriction fragment length polymorphism (RFLP) may also be used. In some embodiments, the somatic mutation is determined by exome sequencing.

In some embodiments, antibodies are raised against the expressed proteins of somatic nucleotide mutations in tumors and used to detect the presence of mutations in such tumors by known techniques, such as enzyme-linked immunosorbent assay (ELISA).

In other embodiments, allele-specific oligonucleotides, or other exquisitely sensitive nucleic acid hybridization probes, may be used to detect specific mutations for use with the method of the present application.

In certain embodiments primers are used to support sequencing of DNA or RNA extracted from a tumor tissue sample. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription.

In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. In other embodiments primers can be extended using isothermal techniques. In some embodiments, techniques and conditions are optimized for the amplification of the ERCC2 gene.

Biological samples used for determining the presence of somatic mutation associated with nucleotide excision repair may be surgically removed tissue samples collected from tumors within the patient, such as, for example, urothelial carcinoma. Tumor tissue samples may be obtained by biopsies, typically using a needle which may or may not have image guidance by, for example, a surgical endoscope. Tumor tissue samples may include samples removed from tumors of colon, lung, ovarian, stomach, renal, uterine, head and neck squamous, and cervical cancer.

In some embodiments, biological sample from the patient is a biopsy sample of muscle-invasive urothelial carcinoma. In some embodiments, the presence of one or more mutations in the ERCC2 gene in the biopsy sample indicates a sensitivity to the treatment by a platinum-based antineoplastic agent, such as cisplatin. In some embodiments, the method further comprises the step of administering to the patient an effective amount of a platinum-based antineoplastic agent or an alkylating antineoplastic agent if one or more mutations associated with nucleotide excision repair are found in the biological sample.

In some embodiments, the somatic mutation determination is performed on biopsies that are embedded in paraffin wax. Formalin fixation and tissue embedding in paraffin wax is a universal approach for tissue processing prior to light microscopic evaluation. A major advantage afforded by formalin-fixed paraffin-embedded (FFPE) specimens is the preservation of cellular and architectural morphologic detail in tissue sections. The use of FFPE specimens provides a means to improve current diagnostics by accurately identifying the major histological types, even from small biopsies. Since FFPE sample collection and storage is a routine practice in pathology laboratories, this approach allows analysis of detection of gene mutations in archived tissues to retrospectively determine sensitivity to platinum-based antineoplastic agents or an alkylating antineoplastic agents.

In some embodiments, the absence of somatic ERCC2 mutation indicates that the urothelial carcinoma is likely to be unresponsive to cisplatin chemotherapy, and the patient then undergoes surgery to remove the carcinoma without accompanying cisplatin chemotherapy. The presence of somatic ERCC2 mutation indicates that the uroethelial carcinoma is likely to be responsive to cisplatin chemotherapy and the patient then undergoes surgery to remove the carcinoma accompanied by cisplatin chemotherapy.

Another aspect of the present application relates to a method of predicting the long-term prognosis of a patient suffering from cancer. The method comprises the steps of determining the presence in a biological sample from the patient of somatic mutation associated with nucleotide excision repair, wherein the presence of a somatic mutation associated with nucleotide excision repair is indicative that platinum-based chemotherapy should be selected for treatment of the patient.

The presence in a biological sample from a patient of somatic mutation associated with nucleotide excision repair, in particular the presence of a somatic ERCC2 mutation, may be detected by any of the standard molecular biological techniques used to detect the presence of a nucleotide mutation, including those listed herein.

If the presence of a somatic ERCC2 mutation is found within the tumor tissue sample from the patient, a superior long-term prognosis may be predicted if a regimen of platinum-based chemotherapy is prescribed for the patient. If the presence of a somatic ERCC2 mutation is not found within the tumor tissue, then a platinum-based chemotherapy will not be selected.

### Method for Treating Cancer

Another aspect of the application relates to a method of treating a patient suffering from cancer. The method comprising the steps of determining the presence in a biological sample from the patient of somatic mutation associated with nucleotide excision repair gene, and administering an effective amount of a platinum-based chemotherapeutic agent or an alkylating antineoplastic agent to the patient if a somatic nucleotide excision repair mutation is detected in the sample.

In some embodiments, the somatic mutation is associated with the ERCC2, ERCC3 or ERCC5 gene. In some embodiments, the somatic mutation is associated with the ERCC2 gene. In some embodiments, the mutation occurs at highly conserved amino acid positions within the helicase domains of ERCC2. In other embodiments, the patient is suffering from urothelial carcinoma, lung cancer (squamous and adenocarcinoma), head and neck squamous carcinoma, cervical cancer, colorectal cancer, esophagogastric cancer, prostate cancer or sarcoma.

In some embodiments, an effective amount of one or more platinum-based chemotherapeutic agent is administered. The each platinum-based chemotherapeutic agent may be administered at a dose of 0.05-500 mg/m² per cycle, 0.05-0.2 mg/m² per cycle, 0.05-0.5 mg/m² per cycle, 0.05-2 mg/m² per cycle, 0.05-5 mg/m² per cycle, 0.05-20 mg/m² per cycle, 0.05-50 mg/m² per cycle, 0.05-100 mg/m² per cycle, 0.05-200 mg/m² per cycle, 0.2-0.5 mg/m² per cycle, 0.2-2 mg/m² per cycle, 0.2-5 mg/m² per cycle, 0.2-20 mg/m² per cycle, 0.2-50 mg/m² per cycle, 0.2-100 mg/m² per cycle, 0.2-200 mg/m² per cycle, 0.2-500 mg/m² per cycle, 0.5-2 mg/m² per cycle, 0.5-5 mg/m² per cycle, 0.5-20 mg/m² per cycle, 0.5-50 mg/m² per cycle, 0.5-100 m g/m² per cycle, 0.5-200 mg/m² per cycle, 0.5-500 mg/m² per cycle, 2-5 mg/m² per cycle, 2-20 mg/m² per cycle, 2-50 mg/m² per cycle, 2-100 mg/m² per cycle, 2-200 mg/m² per cycle, 2-500 mg/m² per cycle, 5-20 mg/m² per cycle, 5-50 mg/m² per cycle, 5-100 mg/m² per cycle, 5-200 mg/m² per cycle, 5-500 mg/m² per cycle, 20-50 mg/m² per cycle, 20-70 mg/m² per cycle, 20-100 mg/m² per cycle, 20-200 mg/m² per cycle, 20-500 mg/m² per cycle, 50-70 mg/m² per cycle, 50-100 mg/m² per cycle, 50-200 mg/m² per cycle, 50-500 mg/m² per cycle, 70-100 mg/m² per cycle, 70-150 mg/m² per cycle, 70-200 mg/m² per cycle, 70-300 mg/m² per cycle, 70-400 mg/m² per cycle, 70-500 mg/m² per cycle, 100-150 mg/m² per cycle, 100-200 mg/m² per cycle, 100-300 mg/m² per cycle, 100-400 mg/m² per cycle, 100-500 mg/m² per cycle, 200-300 mg/m² per cycle, 200-400 mg/m² per cycle, 200-500 mg/m² per cycle, 300-400 mg/m² per cycle, 300-500 mg/m² per cycle and 400-500 mg/m² per cycle. In some embodiments, the platinum-based chemotherapeutic agent is administered at about 10, 20, 30, 40, 50, 60, 70, 80, 90 or 100 mg/m² per cycle. Each cycle may have a length of 1, 2, 3, 4, 5, 6, 7, 8 9 or 10 days, or 1, 2, 3, 4, 5, 6, 7, 8 or 9 weeks.

The platinum-based chemotherapeutic agent may be administered parentally, intravenously, intra muscularly, subcutaneously, or orally.

In some embodiments, the platinum-based chemotherapeutic agent is cisplatin and is administered at a 3-4 week cycle at a dose of 20-100 m g/m² per cycle, 20-70 mg/m² per cycle, 50-70 mg/m² per cycle, 50-100 mg/m² per cycle or 70-100 mg/m² per cycle. The dose range for cisplatin chemotherapy as part of the methods disclosed herein ranges between 20 mg/m² to 100 mg/m² cisplatin. In some embodiments, cisplatin is given parenterally at approximately at 20 mg/m² every 3 to 4 days. In some embodiments, cisplatin at the above described dose is given by IV infusion over 6-8 hours. Cisplatin is commercially available from many sources. The dose to be administered to a subject having a cancer can be determined by a physician based on the subject's age, and physical condition, the sensitivity of the cancer to an antineoplastic agent the nature of the cancer and the stage and aggressiveness of the cancer. In some embodiments, other platinum-based c chemotherapeutic agent, such as carboplatin or oxaliplatin, is given at a dosage in the range approximately 5 mg/m² and 500 mg/m² every 2 days to one week. The dosage ranges herein are not intended to limit the scope of the invention in any way. In some instances dosage levels below the lower limit of the aforesaid dose range may be more than adequate, while in other cases still larger doses may be employed without causing any harmful side effect.

In other embodiments, a platinum-based chemotherapeutic agent, such as cisplatin, is administered in conjunction with surgery that removes the cancer tissue containing a somatic nucleotide excision repair mutation. In some embodiments, the platinum-based chemotherapeutic agent is administered before surgery and after surgery. In other embodiments, the platinum-based chemotherapeutic agent is administered after surgery. In other embodiments, a platinum-based chemotherapeutic agent, such as cisplatin, is administered before, after or in conjunction with radiation therapy.

In some embodiments, a combination of platinum-based chemotherapeutic agents, such as cisplatin/paclitaxel, cisplatin/gemcitabine, or cisplatin/docetaxel is administered.

Somatic mutations associated with nucleotide excision repair may be identified by standard molecular biological techniques used to detect the presence of specific biomarkers in a biological sample, such as a tumor tissue sample. Techniques include the use of primers and probes, which are capable of interacting with the known DNA sequence of nucleotide excision repair genes, such as ERCC2, ERCC3 or ERCC5. Platinum-based chemotherapy uses a specific class of highly cytotoxic chemotherapy drugs that contain platinum as part of a coordination complex. Platinum-based chemotherapy drugs are generally used against advanced, metastatic forms of cancer, such as colon cancer, breast cancer, small cell and non-small cell lung cancer, adrenocortical cancer, anal cancer, endometrial cancer, non-Hodgkin lymphoma, ovarian cancer, testicular cancer, melanoma and head and neck cancers. Platinum-based chemotherapy may be used combination with other kinds of chemotherapy, including agents such as taxanes or anthracyclines. Cisplatin is the most commonly known platinum-based chemotherapeutic agent, however, other platinum-based chemotherapeutic agents include carboplatin, oxaliplatin, satraplatin, picoplatin, nedaplatin, triplatin and lipoplatin.

Surgery upon the tumor in the patient may be carried out in conjunction with the platinum-based chemotherapy or after a regimen of platinum-based chemotherapy has been completed.

### Kits

Another aspect of the present application relates to a kit for practicing the methods of the application. Kits of the application may supply the means to detect a somatic mutation associated with nucleotide excision repair in a biological sample obtained from a tumor in a patient who is a candidate for platinum-based chemotherapy. In some embodiments, the kit is a package or a container comprising one or more reagents for specifically detecting a somatic mutation associated with a nucleotide excision repair gene. In some embodiments, the one or more reagents comprise two or more nucleotide primers or probes that specifically hybridize to one or more nucleotide excision repair genes. In some embodiments, the kit comprises two or more nucleotide primers or probes that specifically hybridize to human ERCC2 gene. In other embodiments, the kit comprises a package insert describing the kit and methods for its use.

In some embodiments, the kits may supply a variety of components including: (1) containers for processing biological samples to obtain nucleotide molecules, in particular DNA and/or RNA (DNA/RNA); (2) reagents for processing biological samples to obtain nucleotide molecules, such as DNA and/or RNA; (3) DNA and/or RNA purification and filtration components, such as microbeads; (4) reagents for DNA and/or RNA filtration and purification; (5) primers and/or other synthetic oligonucleotides to be used for PCR amplification of DNA or other molecular biology techniques for nucleotide sequence analysis, including DNA sequencing; (6) microarrays designed for nucleotide sequence analysis, including hybrid capture arrays; (7) means by which nucleotide sequences may be visualized, including software programs.

In some embodiments, the kit may comprise: (1) synthetic oligonucleotides to be used for PCR amplification of DNA containing somatic ERCC2, ERCC3 or ERCC5 mutations; (2) means by which nucleotide sequences of somatic ERCC2, ERCC3 or ERCC5 mutations may be visualized. A kit may further comprise: a hybrid capture microarray capable of identifying the presence of at least one somatic ERCC2, ERCC3, or ERCC5 mutation in a biological sample from a patient suffering from urothelial carcinoma.

The foregoing descriptions of specific embodiments of the present application have been presented for purposes of illustration and description. They are not intended to be exhaustive or to limit the application and method of use to the precise forms disclosed. Obviously many modifications and variations are possible in light of the above teaching. It is understood that various omissions or substitutions of equivalents are contemplated as circumstance may suggest or render expedient, but is intended to cover the application or implementation without departing from the spirit or scope of the claims of the present application.

### EXAMPLES

### Example 1

### Material and Method

### Patients and Samples

Patients with muscle invasive or locally advanced urothelial carcinoma and extreme responses to chemotherapy (defined as no residual invasive carcinoma at cystectomy or presence of persistent muscle invasive or extravesical disease at cystectomy), available pre-chemotherapy tumor tissue, and enrolled on Institutional Review Board (IRB) approved tissue acquisition and DNA sequencing protocols were identified (Dana-Farber protocols 02-021 and 11-334; Memorial Sloan-Kettering Cancer Center protocols 89-076 and 09-025). All patients provided written informed consent for genomic testing utilized for this study. Specimens were evaluated by genitourinary pathologists to identify tumor-bearing areas for DNA extraction. The minimum percentage of neoplastic cellularity for regions of tumor tissue was 60%. Study specimens of frozen or formalin-fixed, paraffin-embedded (FFPE) tissue sections were identified at the Dana-Farber Cancer Institute and Memorial Sloan-Kettering Cancer Center. Germline DNA was extracted either from peripheral blood mononuclear cells or histologically normal non-urothelial tissue.

TABLE 1
Patient Characteristics

| | | | Non- | |
|---|---|---|---|---|
| | Total | Responders | Responders | |
| | (N = 50) | (N = 25) | (N = 25) | |
| Age at TUR - yr | 62.5 ± 8.9 | 61 ± 10.1 | 64 ± 7.3 | p > 0.05 |
| Female sex - no. (%) | 13 (26) | 6 (24) | 7 (28) | |
| Ethnicity - no. (%) | | | | |
| Hispanic/Latino | 1 (2) | 0 (0) | 1 (4) | |
| Non- | 48 (96) | 24 (96) | 24 (96) | |
| Hispanic/Non- | | | | |
| Latino | | | | |
| Unknown | 1 (2) | 1 (4) | 0 (0) | |
| Race - no. (%) | | | | |
| Caucasian | 49 (98) | 25 (100) | 24 (96) | |
| African-American | 1 (2) | 0 (0) | 1 (4) | |
| Smoking Status - no. | | | | |
| (%) | | | | |
| Never | 13 (26) | 8 (32) | 5 (20) | |
| Former | 26 (52) | 13 (52) | 13 (52) | |
| Current | 11 (22) | 4 (16) | 7 (28) | |
| Clinical Staging - no. | | | | |
| (%) | | | | |
| T2 | 37 (74) | 19 (76) | 18 (72) | |
| T3 | 10 (20) | 5 (20) | 5 (20) | |
| T4 | 3 (6) | 1 (4) | 2 (8) | |
| N0 | 40 (80) | 18 (72) | 22 (88) | |
| Node Positive | 10 (20) | 7 (28) | 3 (12) | |
| TUR Histology - no. (%) | | | | |
| TCC | 32 (64) | 19 (76) | 13 (52) | |
| Mixed TCC | 18 (36) | 6 (24) | 12 (48) | |
| Neoadjuvant | | | | |
| Chemotherapy Regimen - | | | | |
| no. (%) | | | | |
| GC | 31 (62) | 14 (56) | 17 (68) | |
| ddMVAC | 16 (32) | 9 (36) | 7 (28) | |
| GC-Sunitinib | 2 (4) | 2 (8) | 0 (0) | |
| ddGC | 1 (2) | 0 (0) | 1 (4) | |
| Median interval from | 47 ± 29.9 | 46 ± 26.0 | 47 ± 33.8 | p > 0.05 |
| chemotherapy to | | | | |
| cystectomy (Days ± SD) | | | | |
| Median length of follow- | 351 ± 363.2 | 372.5 ± 416.2 | 329.5 ± 287.1 | p > 0.05 |
| up (Days ± SD)* | | | | |
| Patients with | 13 (26) | 2 (8) | 11 (44) | |
| Recurrence - no. (%) | | | | |

Clinical characteristics of the total patient cohort, along with data stratified by responder or non-responder status.
*For patients alive at the time of this study only. P < 0.05 is considered significant (Mann-Whitney Test). (TUR: transuretheral resection; GC: gemcitabine and cisplatin; ddMVAC: dose dense MVAC; ddGC: dose dense gemcitabine and cisplatin; SD: standard deviation).
Plus-minus values are medians ± standard deviation.

### Whole Exome Sequencing and Statistical Analysis

DNA extraction and exome sequencing: slides were cut from FFPE or frozen tissue blocks and examined by a board-certified pathologist to select high-density cancer foci and ensure high purity of cancer DNA. Biopsy cores were taken from the corresponding tissue block for DNA extraction. DNA was extracted using Qiagen's QIAamp DNA FFPE Tissue Kit Quantitation Reagent (Invitrogen). DNA was stored at −20 degrees Celsius. Whole exome capture libraries were constructed from 100 ng of DNA from tumor and normal tissue after sample shearing, end repair, and phosphorylation and ligation to barcoded sequencing adaptors. Ligated DNA was size selected for lengths between 200-350 bp and subjected to exonic hybrid capture using SureSelect v2 Exome bait (Agilent). The sample was multiplexed and sequenced using Illumina HiSeq technology for a mean target exome coverage of 121× for the tumors and 130× for germline samples. Four cases did not complete the exome sequencing process due to sequencing process failure.

Sequence data processing: exome sequence data processing and analysis were performed using pipelines at the Broad Institute. A BAM file aligned to the hg19 human genome build was produced using Illumina sequencing reads for the tumor and normal sample and the Picard pipeline. BAM files were uploaded into the Firehose infrastructure (http://www.broadinstitute.org/cancer/cga/Firehose), which managed intermediate analysis files executed by analysis pipelines.

Sequencing quality control: sequencing data was incorporated into quality control modules in Firehose to compare the tumor and normal genotypes and ensure concordance between samples. Cross-contamination between samples from other individuals sequenced in the same flow cell was monitored with the ContEst algorithm. Samples with >4% contamination were excluded (n=4).

Alteration identification and annotation: the MuTect algorithm was applied to identify somatic single-nucleotide variants in targeted exons. Indelocator was applied to identify small insertions or deletions (http://www.broadinstitute.org/cancer/cga/indelocator). Gene level coverage was determined with the DepthOfCoverage in the Genome Analysis Tool Kit. Alterations were annotated using Oncotator (http://www.broadinstitute.org/cancer/cga/oncotator). Power calculations for coverage were determined using the MuTect coverage file, which requires a minimum of 14× coverage in the tumor. Samples with median allelic fractions less than 0.05 and insufficient DNA for orthogonal validation with Fluidigm Access Array were excluded (n=1). Alteration significance: MutSigCV²⁰ was applied to the aggregate cohort of 50 cases to determine statistically altered genes in the cohort. Alterations from all nominated significant genes from MutSigCV were manually reviewed in the Integrated Genomics Viewer (IGV). Alterations that were invalid based on IGV review (as a result of misalignment artifacts viewable in IGV) were subsequently excluded from the final results, which resulted in the exclusion of TGFBR1 and DEPDC4 from the final result.

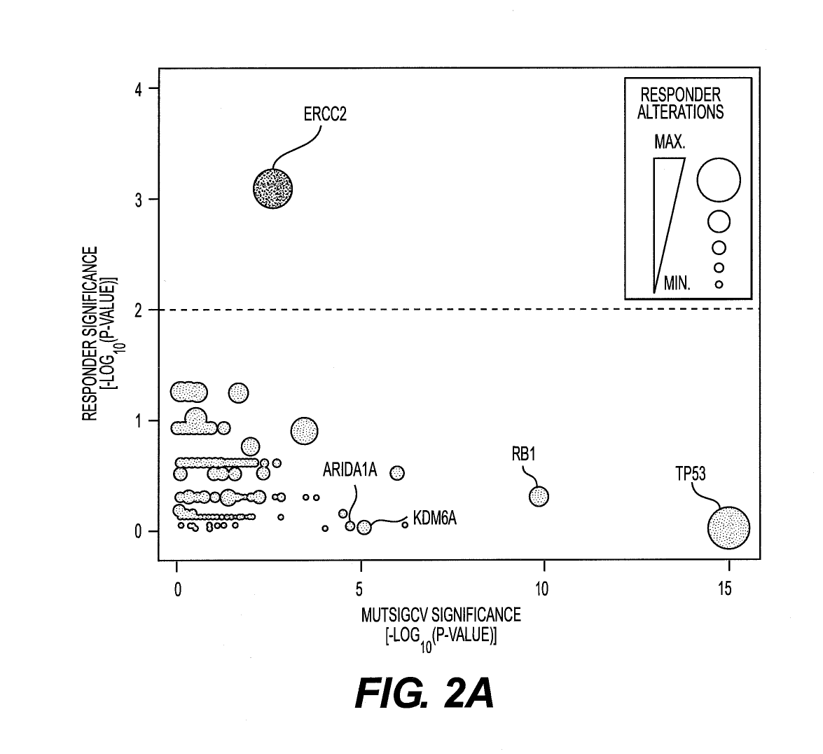

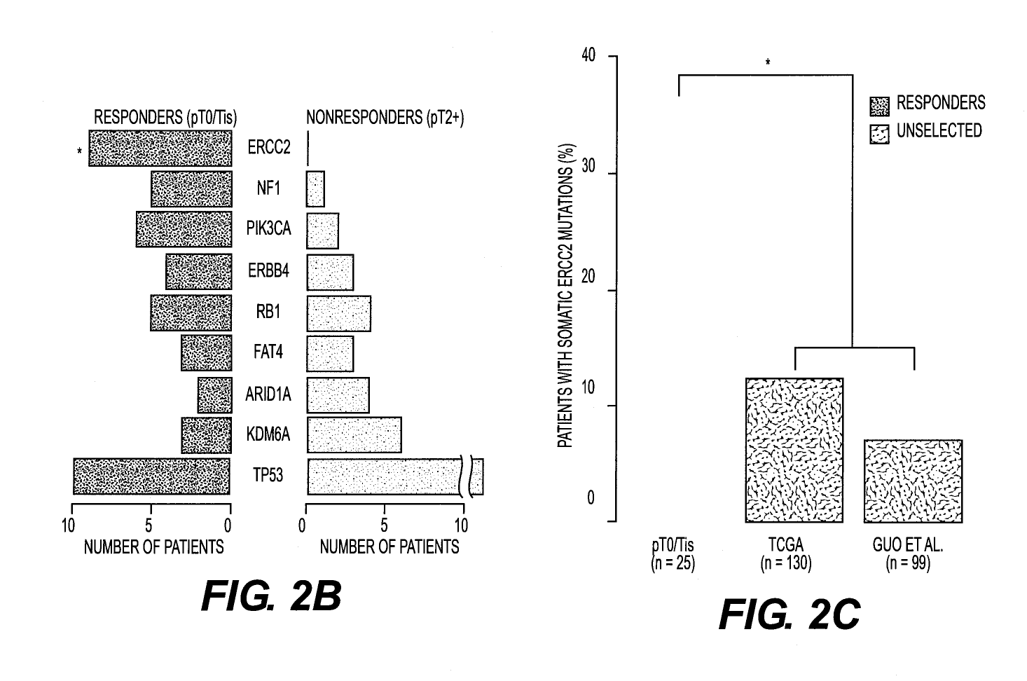

Selective gene enrichment analysis: all somatic mutations and short insertion/deletions were aggregated for the cohort and split between responders and non-responders. Alterations with coverage at the tumor site of >=30× and an allelic fraction >=0.1 were considered for further analyses. Missense, nonsense, and splice site mutations, along with short insertion/deletions, were then assigned a damaging score (range 0-1) following previously reported methods: missense mutations were scored using the Polyphen2 score for the amino acid substitution. Missense mutations without available Polyphen2 scores (due to mapping errors or dinucleotide status) were listed as “Unavailable” and excluded. Nonsense mutations, splice site mutations, and short insertion/deletions were assigned a damaging score of 1. Alterations with a damaging score ≧0.5 were then tabulated for occurrence in responders and non-responders. An altered gene would only be counted once per patient. Fisher's exact test was performed to compare between cohorts to derive a p-value for each gene. Since a minimum of 6 alterations were required to observe a p-value of <=0.01, only genes with >=6 alterations in the cohort (thereby representing >10% of the cohort and of highest potential clinical significance) were considered for multiple hypothesis testing. Comparison of ERCC2 mutation frequency in the responders was compared to the unselected TCGA and Chinese cohorts with a binomial test. Results from this analysis are made available in FIG. 2C. Comparison of ERCC2 mutation distribution between responders and non-responders adjusted for elevated mutation rate in the responders was performed with the binomial test conditional on observing 9 mutations and using estimated ratio of mutation rates between responders and non-responders as the expected frequency of ERCC2 mutations in responders under the null hypothesis (e.g. for ERCC2:
binom.test(x=9, n=9, p=Mutation_Rate^{Responders}/Mutation_Rate^{Non-Responders}, alternative=“greater”)).

All statistical calculations were performed using the R statistical package.

### Mutation Validation

Orthogonal validation of selected mutations and short insertion/deletions (those presented in this manuscript, including ERCC2) was performed using the Fluidigm Access Array. Of 50 cases, 35 had sufficient DNA to generate sufficient read depth for analysis. A total of 85 candidate targets were submitted to Fluidigm for single-plex PCR primer assay design. This resulted in the design of 65 assays covering all 85 targets. Assay amplicons ranged from 163 bp to 199 bp in size, with an average of 183 bp. All available samples, were run on the Access Array system (Fluidigm) using three 48.48 Access Array IFC chips following manufacturer's recommendations using the ‘4-Primer Amplicon Tagging protocol’ for Access Array (Fluidigm, P/N 100-3770, Rev. C1) with the exception that Access Array IFC chips were loaded and harvested using a Bravo Automated Liquid Handling Platform (Agilent Technologies), using manufacturer's recommendations. Resulting amplicons containing sample specific barcodes and Illumina adapter sequencers were pooled and sequenced on a MiSeq sequencer (Illumina) with 2 runs of 150 base paired-end reads (V2 sequencing chemistry), using custom Fluidigm sequencing primers following manufacturer's recommendations (Fluidigm). All sites were manually reviewed in IGV to determine presence or absence of non-reference reads. Details about validation results for ERCC2 are in Table 2. Variants where there was inadequate sample for validation or insufficient reads to interpret manually in IGV were listed as “Unavailable”.

### Cloning and Cell Lines

A site-directed PCR mutagenesis/BP recombination method was used to generate WT and mutant ERCC2 open reading frames (ORFs). For each mutant, PCR products were generated such that fragments overlap at the region of the desired mutation. The fragments were then introduced into the pDONR vector through BP reaction. The BP reaction mixture was transformed into E. coli and recombined to generate a pENTR vector. The pENTR vector was then used to perform the LR reaction to create an expression plasmid.

The expression plasmids harboring WT ERCC2, GFP (negative control), or mutant ERCC2s were expanded in E. coli TOP10 cells (Invitrogen) and purified using an anion exchange kit (Qiagen). Lentiviruses were propagated in 293T cells by cotransfection of the expression plasmid with plasmids encoding viral packaging and envelope proteins. Unless otherwise noted, all human cell lines were cultured in Dulbecco's Modified Eagle's Medium (Invitrogen) supplemented with 10% fetal bovine serum (Sigma) and 1% L-glutamine and grown at 37° C. and 5% CO₂. The 293T cell supernatants containing virus were collected after 48 hours, filtered twice (0.45 μm syringe filter, Millipore), then added directly to growing cultures of an SV40-transformed pseudodiploid ERCC2-deficient fibroblast cell line derived from an XP patient of genetic complementation group D (GM08207; Coriell Institute). Polybrene® (Sigma) was added to a final concentration of 8 μg/ml to increase the efficiency of infection. Stable integrates were selected by incubation for 5 days in media containing 10 μg/ml blastocidin. Physical and biologic containment procedures for recombinant DNA followed institutional protocols in accordance with the National Institutes of Health Guidelines for Research involving Recombinant DNA Molecules.

### Cisplatin Sensitivity Assays

Cells were transferred to 96-well plates at a density of 500 cells per well. Cisplatin (Sigma) was serially diluted in media and added to the wells. After 120 hours, CellTiter-Glo reagent (Promega) was added to the wells and the plates were scanned using a luminescence microplate reader (BioTek). Survival at each cisplatin concentration was plotted as a percentage of the survival in cisplatin-free media. Each data point on the graph represents the average of three measurements, and the error bars represent the standard deviation. IC₅₀ concentrations were calculated using a four parameter sigmoidal model and plots were generated using Prism (GraphPad). A one-way ANOVA test was used to compare the IC₅₀ of the negative control cell line to the IC₅₀ of the WT and mutant ERCC2 cell lines, and to compare the IC₅₀ of the WT line to the IC₅₀ of the negative control and mutant cell lines.

### UV Clonogenic Survival Assays

Cells were seeded in 6-well plates (Nunc) at a density of 1500 cells per well. The following day, the cells were washed once and then exposed to increasing UV doses using a UV-B irradiator (Stratagene). Media was replaced and the cells were allowed to grow for nine days. On day 10, cells were fixed using a 1:5 acetic acid:methanol solution for 20 minutes at room temperature. Cells were then stained for 45 minutes using 1% crystal violet in methanol solution. Plates were rinsed vigorously with water, allowed to dry, and colonies were then manually counted. The number of colonies present at each UV dose was plotted as a ratio of the number of colonies present in mock-irradiated wells. Each data point represents the average of at least three measurements, and the error bars represent the standard deviation.

### Chromosomal Breakage Analysis

Approximately 1×10⁶ cells were seeded per 10 cm dish. After 24 hours, 400 nM cisplatin was added and cells were allowed to grow for an additional 48 hours. Cells were exposed to colcemid for 2 hours, harvested using 0.075 M KCl, and fixed in 3:1 methanol:acetic acid. Slides were stained with Wright's stain and 25-50 metaphases were analyzed.

### Immunoblots

Frozen cell pellets were thawed and resuspended in RIPA buffer (50 mM TRIS [pH 7.3], 150 mM NaCl, 1 mM EDTA, 1% Triton X-100, 0.5% Na-deoxycholate, 0.1% SDS) supplemented with complete protease inhibitor (Roche), NaVO₄, and NaF. The cell suspensions were centrifuged and total protein concentration of the supernatant was determined by colorimetry (Bio-Rad). Samples were boiled with loading buffer (Bio-Rad) and electrophoresed in a 3-8% gradient TRIS-acetate gel (Life Technologies). Resolved proteins were transferred to a PVDF membrane (Millipore) at 90V for 2 hours at 4° C. The membrane was blocked for one hour in blocking solution (5% milk in TRIS-buffered saline-T) and incubated with primary antibody in blocking solution at 4° C. overnight (mouse ERCC2, AbCam; rabbit β-actin, Cell Signaling). The following day, the membrane was rinsed and incubated for one hour with peroxidase-conjugated secondary antibody in blocking solution (anti-mouse and anti-rabbit, Cell Signaling) and rinsed. Enhanced chemiluminescent substrate solution (PerkinElmer) was added and signal was detected by film exposure (GE Healthcare).

### Example 2

### Mutations Associated with Response to Platinum-Based Chemotherapy

Cisplatin-based chemotherapy is the standard of care for patients with muscle invasive urothelial carcinoma. Pathologic downstaging to pT0/pTis after neoadjuvant cisplatin-based chemotherapy is associated with improved survival, although the molecular determinants of cisplatin response are incompletely understood.

Samples initially consisted of patients treated at Dana-Farber, and expanded with samples from Memorial Sloan-Kettering. Tumor material was retrieved from the DFCI urothelial carcinoma repository and the MSKCC bladder tumor tissue bank. Sequencing of tumor and normal material using whole exome sequencing was performed to assess the incidence of somatic and germline genomic alterations.

Whole exome sequencing was performed on pre-treatment tumor and germline DNA from 50 patients with muscle invasive urothelial carcinoma who received neoadjuvant cisplatin-based chemotherapy followed by cystectomy (25 pT0/pTis “responders”, 25 pT2+“non-responders”) to identify somatic mutations that occurred preferentially in cisplatin responders. Somatic ERCC2 mutations were observed in 36% of responders and 0% non-responders (q<0.01). ERCC2, a nucleotide excision repair gene required for efficient repair of DNA crosslinks, was the only significantly mutated gene enriched in the cisplatin responders compared with non-responders. ERCC2 mutations clustered within or near conserved helicase domains. Expression of representative ERCC2 mutations in an ERCC2-deficient cell line failed to rescue cisplatin and UV sensitivity compared to wild-type ERCC2. These results suggest that lack of normal ERCC2 function contributes to cisplatin sensitivity and that ERCC2 mutation status can inform the use of cisplatin-containing regimens in muscle invasive urothelial carcinoma and other ERCC2-mutated tumors.

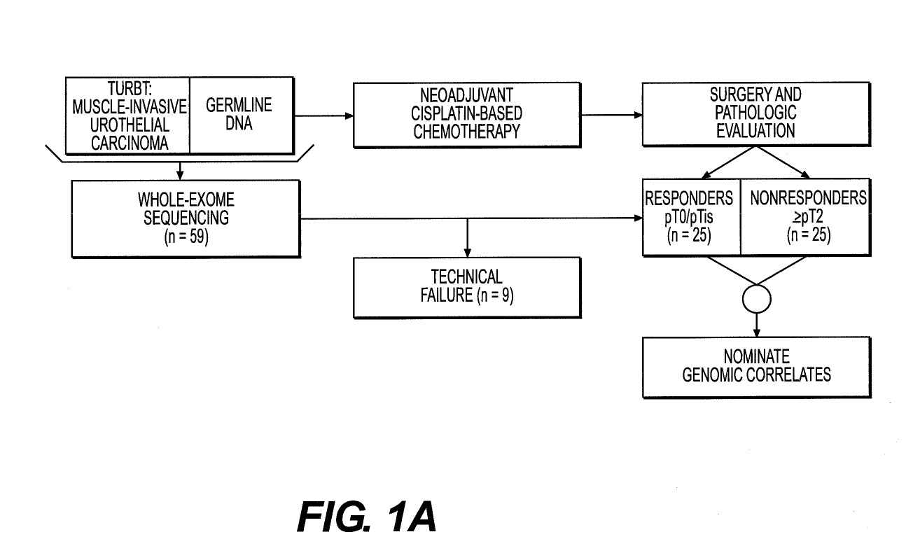

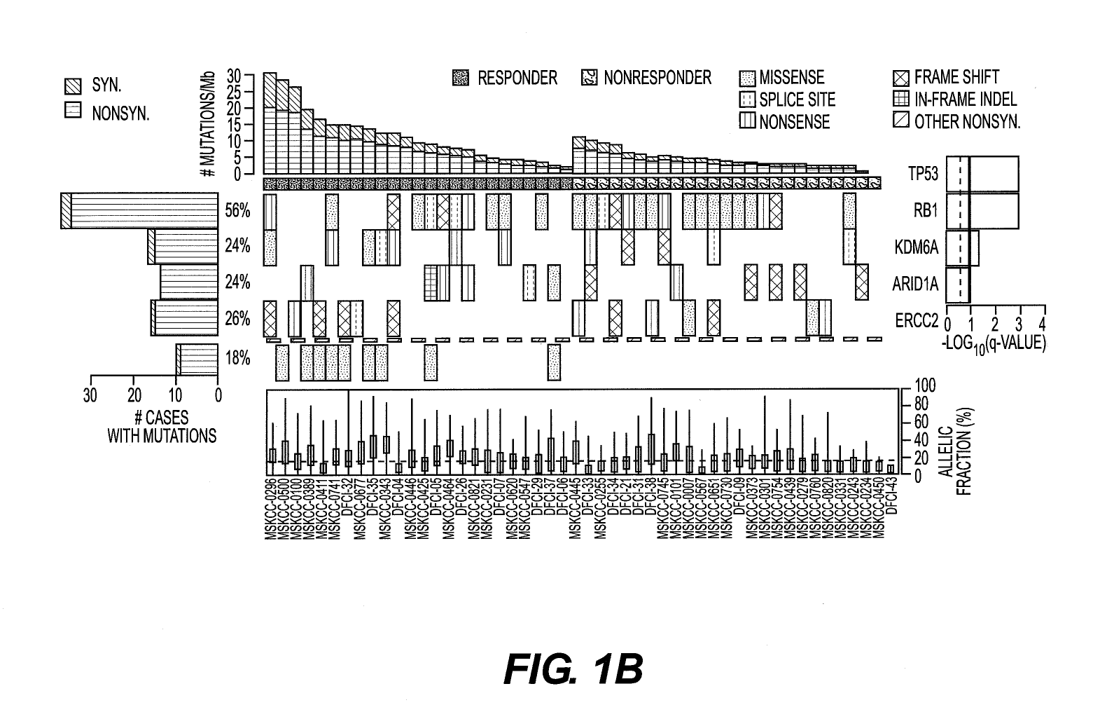

Specifically, tumor and germline DNA was sequenced from 50 patients; 25 (50%) who experienced a pathologic complete response (pT0) or carcinoma in situ (pTis) following neoadjuvant chemotherapy (“responders”), and 25 (50%) with persistent muscle invasive or progressive disease (≧pT2) at cystectomy (“non-responders”) (FIG. 1A). Although multiple chemotherapeutic regimens were utilized, all contained cisplatin (Table 1). No significant differences in clinical characteristics were identified between responders and non-responders at baseline (P>0.05; Mann-Whitney).

The mean target coverage achieved following whole exome sequencing was 121× for tumors and 130× for paired germline samples. The median mutation rate was 9.7 mutations per megabase (mutations/Mb) for responders and 4.4 mutations/Mb for non-responders. The mutation rate for the chemotherapy responders was higher than the non-responders (P=0.0003; Mann-Whitney test) (FIG. 1B), raising the possibility of reduced DNA repair fidelity among the cisplatin responders.

A statistical assessment of the base mutations and short insertion/deletions across both responders and non-responders nominated four genes as significantly altered: TP53, RB1, KDM6A, and ARID1A (FIG. 1B). Somatic alterations in each of these genes have been implicated in urothelial carcinoma. In addition, nine non-synonymous somatic mutations were observed in ERCC2, a member of the NER pathway (FIG. 1B, Table 2). Although this gene did not reach cohort-wide statistical significance, its known role in DNA repair and report of being recurrently mutated in bladder cancer raised the possibility that such mutations might associate with cisplatin response in this cohort.

Relevant somatic and germline alterations were identified by adaptation and modification of discovery-oriented algorithms in common use at the Broad Institute. These identified frequent ERCC2, ERCC3, and ERCC5 mutations (72% in the latest update) in patients with complete responses to platinum-based combination chemotherapy, compared to 0% in patients with primary resistance. The baseline frequencies of these mutations in patients with muscle invasive urothelial carcinoma is approximately 14%, indicating a dramatic enrichment in the responding patients. ERCC2 and ERCC3 (also known as XPD and XBP) are helicases and ATP-aces that make up the core of the TFIIH complex, recognize DNA damage, and are required for nucleotide exicison repair (NER). ERCC5 (XPG) interacts with ERCC2 and stabilizes the TFIIH complex, and incises the DNA 5′ to the damaged bases. These genes are implicated in the autosomal recessive inherited cancer susceptibility syndromes xeroderma pigmentosum (ERCC 2 and ERCC3) and Cockayne syndrome (ERCC5).

An enrichment analysis was performed to nominate genes that were selectively mutated in the responders compared to non-responders. Among 3,277 genes that harbored at least one possibly damaging somatic alteration (missense mutation with a PolyPhen2 score ≧0.5, or any nonsense mutation, splice site mutation, or short insertion/deletion), ERCC2 was the only altered gene significantly enriched in the responder cohort (FIG. 2A). Indeed, all ERCC2 non-synonymous somatic mutations occurred in the cisplatin sensitive tumors (P<0.001; Fisher's exact test). ERCC2 remained significantly enriched in responders following false discovery analysis performed on genes in which the mutation frequency afforded adequate power for statistical assessment (q=0.007; Benjamini-Hochberg) (FIG. 2B).

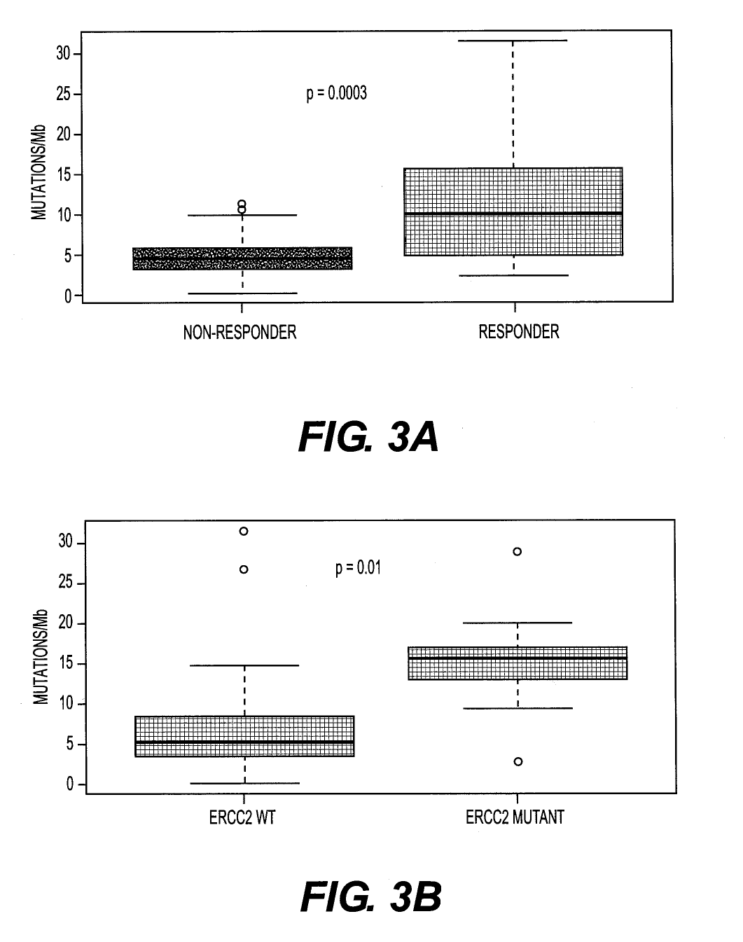

Moreover, the enrichment for ERCC2 mutations in the responder group was also significant when adjusted for differences in overall mutation rate between responders and non-responders (P=0.04; binomial test). Towards this end, the median background mutation rate for ERCC2 mutant tumors (15.5 mutations per megabase) was significantly elevated compared to ERCC2 wild-type tumors (5.1 mutations per megabase) (P=0.01; Mann-Whitney test) (FIG. 3B), consistent with a possible DNA repair defect in these cases and prior reports.

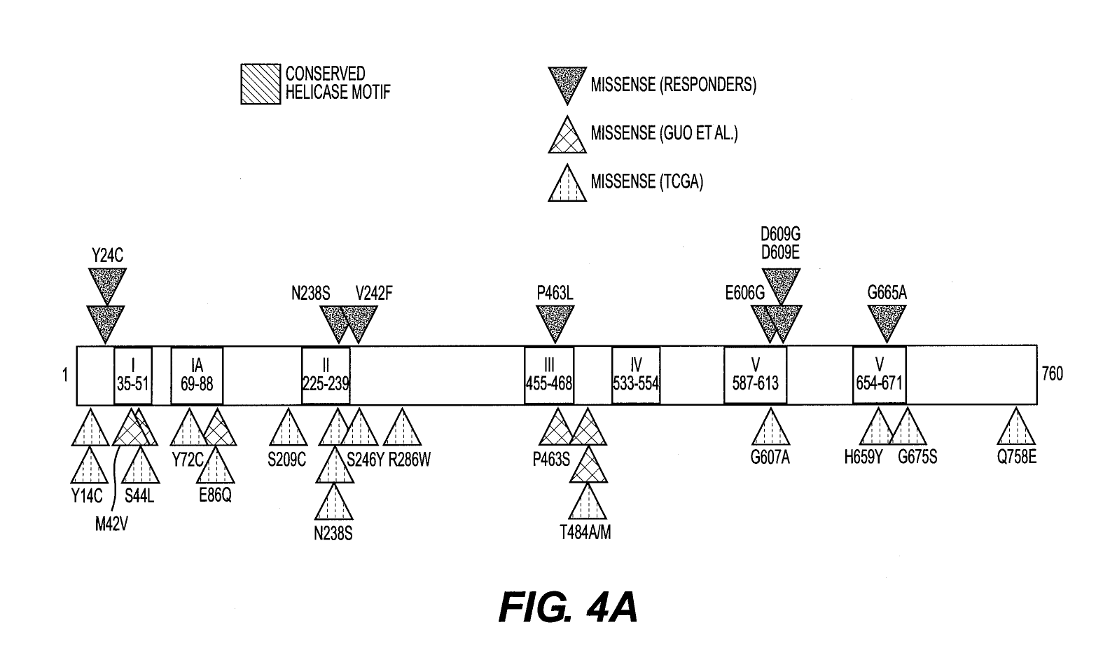

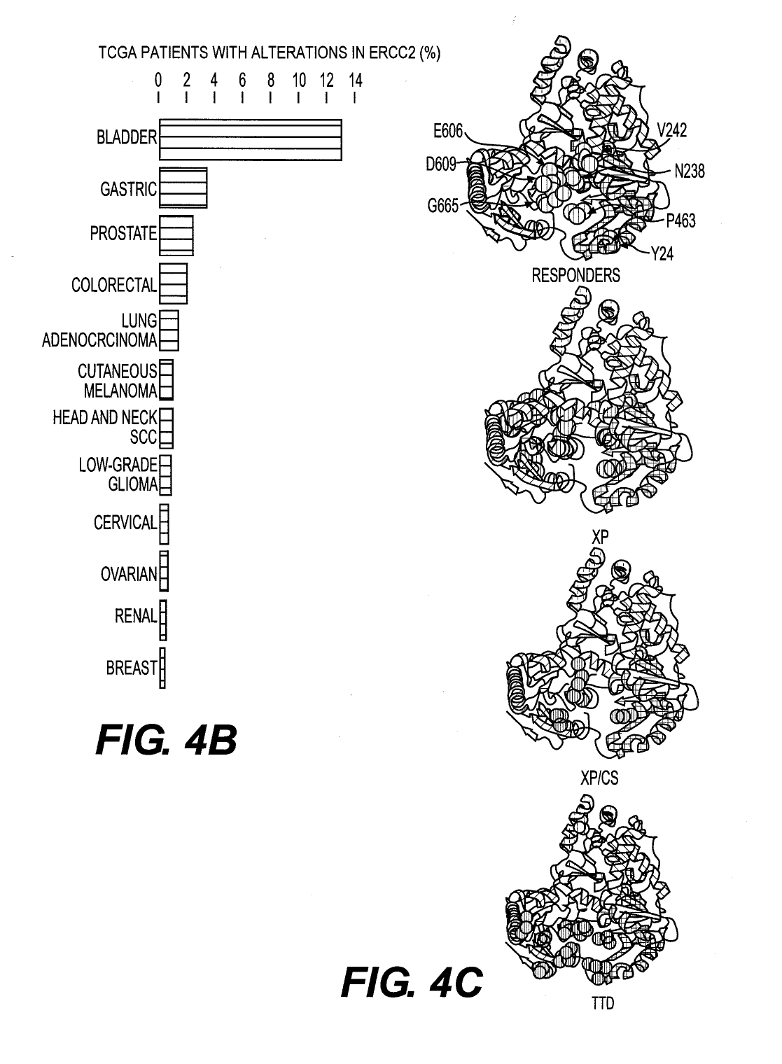

The frequency of ERCC2 mutations in the responder cohort was also compared to the somatic ERCC2 mutation frequency in two unselected bladder cancer populations: 130 cases from the Cancer Genome Atlas (TCGA) project who did not receive neoadjuvant chemotherapy prior to sample acquisition and 99 cases from a Chinese patient cohort (FIG. 4A). Sixteen (12%) TCGA and 7 (7%) Chinese cases harbored somatic ERCC2 mutations. When compared to these unselected populations, ERCC2 was significantly enriched in the cisplatin responder cohort (36% of cases; p<0.001; binomial test) (FIG. 2C).

To determine the relative abundance of somatic ERCC2 mutations in urothelial carcinoma relative to other tumor types, TCGA data from 19 tumor types (n 4,429) was queried. Somatic ERCC2 mutations were observed at low frequencies (<4%) in 11 other tumor types, including several tumor types for which platinum-based chemotherapy is currently a standard part of the treatment pathway (FIG. 4B).

Cisplatin was the only common therapy among the nine chemotherapy-responsive patients whose tumors harbored somatic ERCC2 mutations. Furthermore, all somatic ERCC2 mutations in the responders occurred within or adjacent to conserved helicase motifs when mapped on an archaebacterial ERCC2 crystal structure (FIG. 4A and FIG. 4C). Similarly, germline mutations in patients with XPD and combined Cockayne syndrome (XP/CS), two disorders with impaired NER function, cluster near helicase domains (FIG. 4C). Conversely, mutations causing trichothiodystrophy (TTD), a disease resulting from alteration of ERCC2's normal role in transcription, are distributed throughout the protein.

### Example 3

### Effect of Somatic Nucleotide Excision Repair Mutations

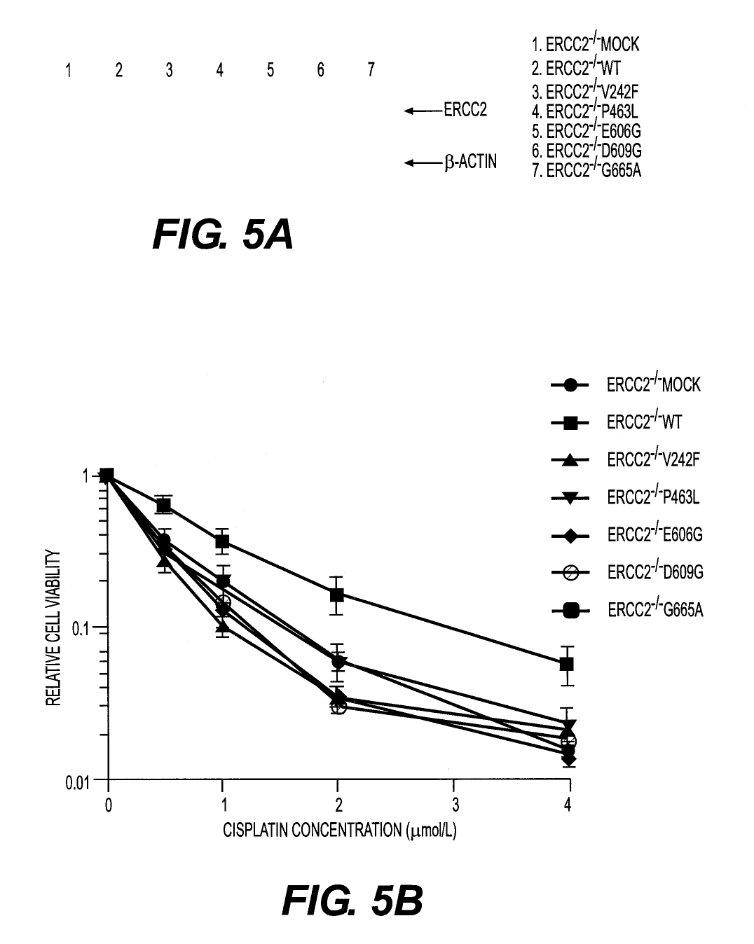

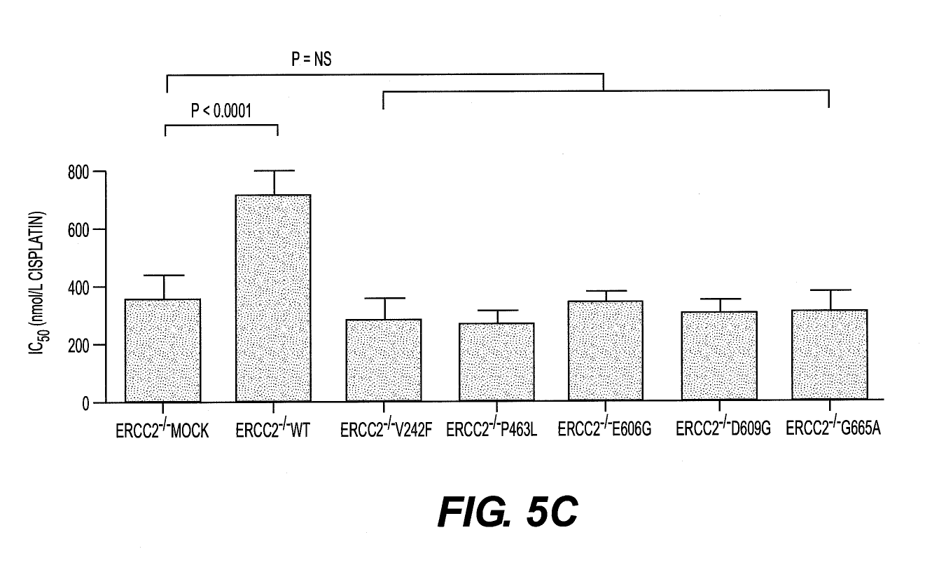

These observations raised the possibility that identified mutations might disrupt ERCC2 helicase function and therefore interfere with NER. To test this hypothesis, five of the identified ERCC2 mutants were stably expressed in an immortalized ERCC2-deficient cell line derived from an XPD patient, and the cisplatin sensitivity profile of each of these cell lines was measured. Expression of wild-type ERCC2 rescued cisplatin sensitivity of the ERCC2-deficient cell line, whereas none of the ERCC2 mutants were able to rescue cisplatin sensitivity (FIG. 5A). The sensitivity curves were used to calculate a cisplatin IC₅₀ for each cell line. The IC₅₀ for the WT ERCC2 complemented cell line was significantly higher than the ERCC2-deficient parent cell line (P=0.0003; ANOVA), whereas the IC₅₀ for each of the mutant ERCC2 cell lines was not significantly different than the parent ERCC2-deficient cell line (FIG. 5B). Similarly, the IC₅₀ for the WT ERCC2 cell line was significantly higher than the IC₅₀ for the ERCC2-deficient and mutant cell lines (p-values <0.0001 to 0.0007; ANOVA).

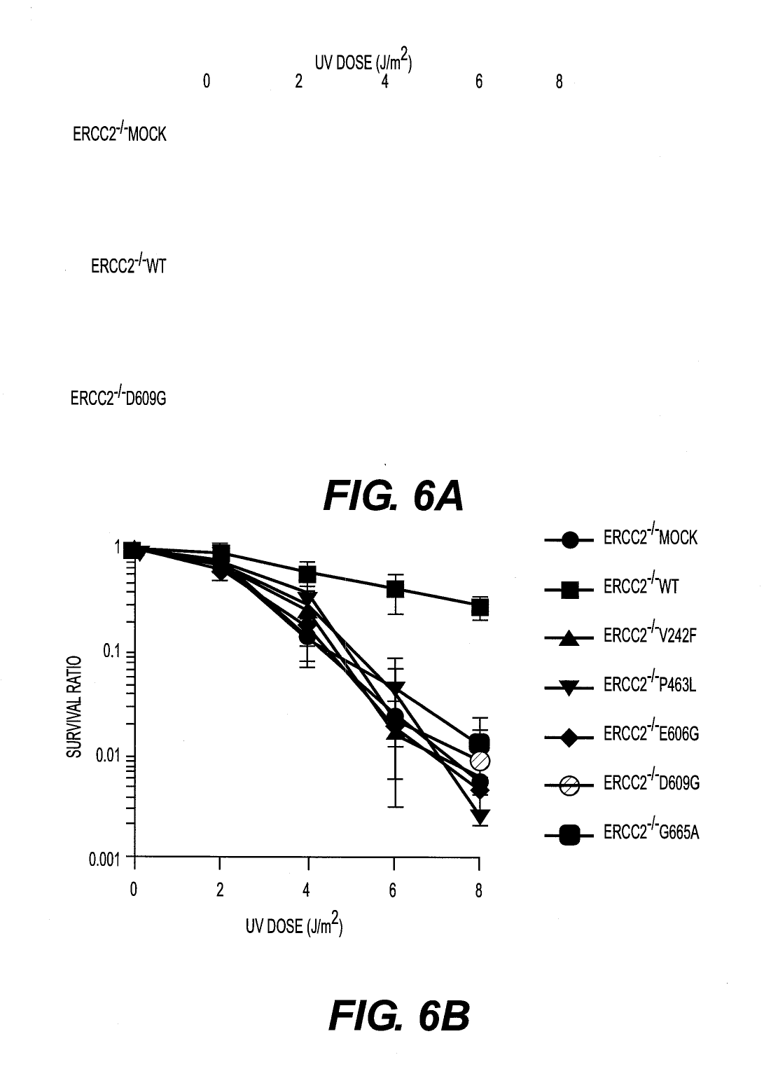

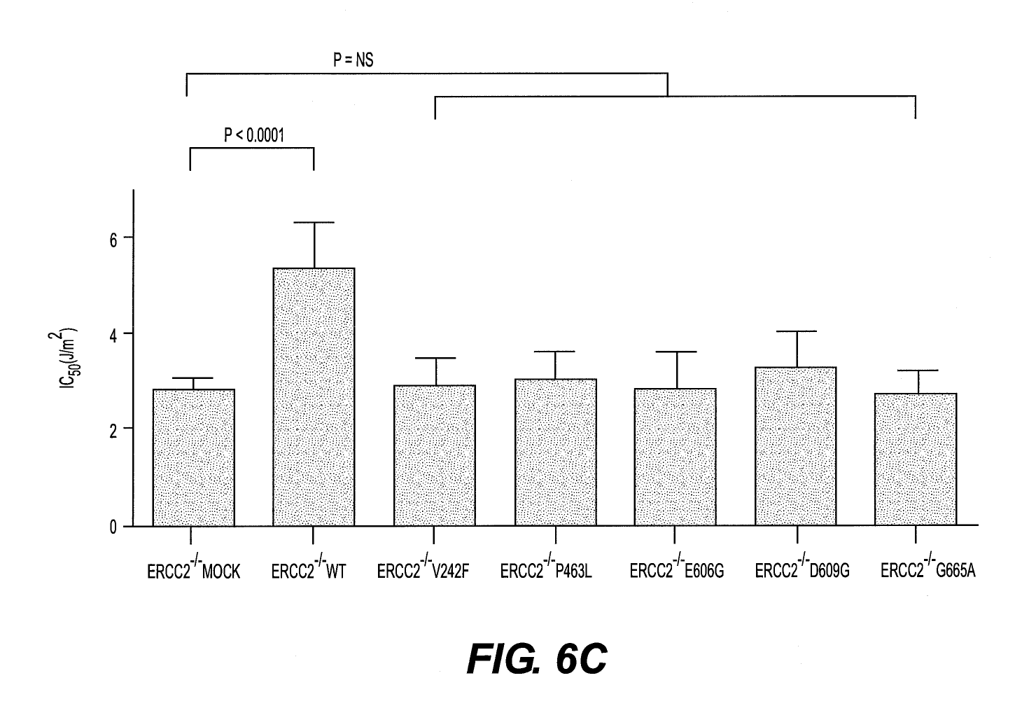

The NER pathway repairs DNA damage beyond cisplatin adducts, and therefore NER function was further interrogated by determining the effect of the identified ERCC2 mutations on NER-mediated repair of UV damage. WT and mutant ERCC2 complemented cell lines were exposed to increasing doses of UV irradiation and clonogenic survival was measured by colony formation. Whereas the WT ERCC2 complemented cell line was able to rescue UV sensitivity of the ERCC2-deficient cell line, the UV sensitivities of the mutant ERCC2 expressing cell lines were not significantly different than that of the ERCC2-deficient cell line (FIGS. 6A-C).

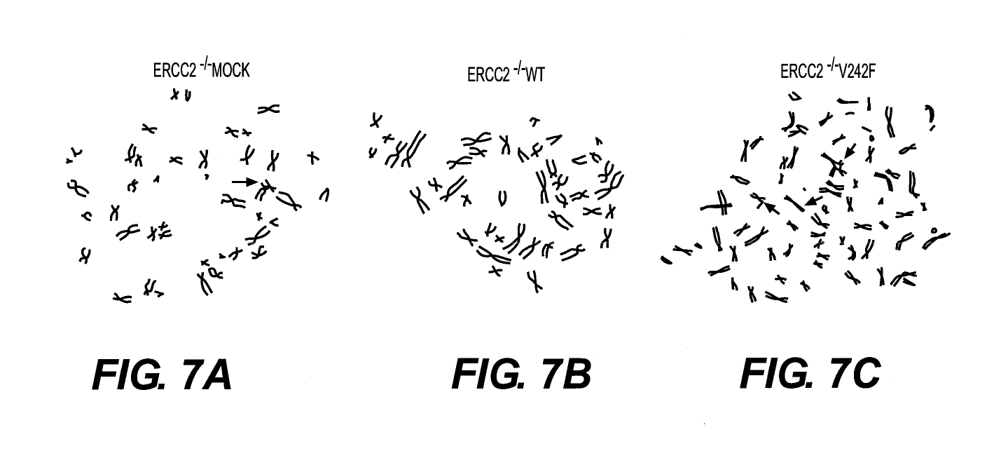

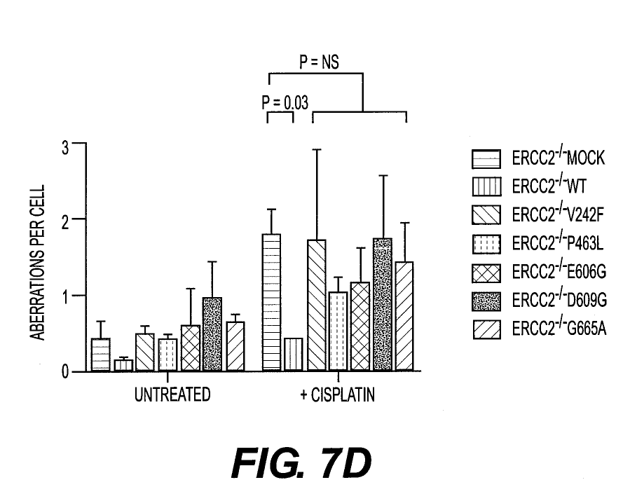

Since the overall mutation rate was higher in ERCC2-mutated tumors than in WT ERCC2 tumors, it may be inferred that ERCC2 mutations may be broadly contributing to genomic instability. Thus, rates of chromosomal aberrations in WT were measured and mutant ERCC2 cell lines before and after cisplatin treatment. Expression of WT ERCC2 in an ERCC2-deficient background results in slightly lower rates of chromosomal aberrations at baseline (FIG. 7D). However, following cisplatin administration, WT ERCC2 expression significant reduces the number of chromosomal aberrations compared to the ERCC2-deficient parent cell line (FIGS. 7A-C). Expression of the identified ERCC2 mutants results in only partial rescue of chromosomal stability, suggesting that the identified ERCC2 mutations are insufficient to maintain genomic stability.

Together, these data demonstrate that the ERCC2 mutations identified in the responder cohort are unable to functionally complement the NER deficiency of an ERCC2-deficient cell line and suggest that the observed cisplatin sensitivity of ERCC2 mutant tumors is due at least in part to loss of normal NER capacity. Furthermore, ERCC2 mutations appear to contribute to genomic instability at both the nucleotide and chromosomal levels, as evidenced by the increased overall mutation rate of ERCC2 mutant tumors and increased chromosomal instability of mutant ERCC2 expressing cell lines.

Overall, these findings show that ERCC2 mutation status can provide a genetic means to select patients most likely to benefit from aggressive cisplatin-based chemotherapy, while directing other patients towards immediate surgery or other novel therapeutic approaches. Subject to further clinical trials, the clinical predictive power of somatic ERCC2 mutation status for cisplatin response will be of great benefit to patients. In addition, since half of patients with bladder cancer are not candidates for cisplatin-based chemotherapy, it indicates that less toxic carboplatin-based neoadjuvant therapies are appropriate for non-cisplatin eligible patients with ERCC2 mutant tumors.

A majority of cases in our cohort still had no recurrent genomic determinant of cisplatin response. It is possible that focal copy number changes, epigenetic or expression-based alterations in DNA repair genes not readily detectable with whole exome sequencing may mediate cisplatin sensitivity in these cases. Also, since approximately one third of patients who achieve pT0 status do so by transurethral resection alone, the relative proportion of ERCC2-mediated cisplatin sensitivity may be higher than observed in this cohort. Preliminary evidence from TCGA also suggests that ERCC2 is less likely to be associated with complete transurethral resection. Additionally, in seven of the nine ERCC2-mutant cases, the ERCC2 mutation allelic fraction was <0.5 (Table 2). The mutations cluster around helicase regions, rather than being randomly distributed throughout the gene as is seen in other tumor suppressors. This suggests that the mutation may be acting through haploinsufficiency or a dominant negative effect to impact NER, rather than as a traditional “two-hit” tumor suppressor model. Further studies may reveal the precise mechanism through which ERCC2 exerts the NER deficiency effect.

In conclusion, this work demonstrates new insights into the relationship between urothelial carcinoma somatic genetic alterations and clinical response to cisplatin-based chemotherapy. These results can inform therapeutic decision-making, novel therapeutic development, and clinical trial designs for neoadjuvant and metastatic urothelial carcinoma. The identification of ERCC2 mutations in other platinum-treated tumor types indicates that they can predict platinum sensitivity in those contexts as well. Finally, these results show that somatic genomic alterations can reveal the mechanistic underpinnings of anti-tumor response to conventional cytotoxic chemotherapy.

1. A method of determining sensitivity to cancer treatment in a patient suffering from cancer, the method comprising the steps of:
determining the presence of somatic mutation in one or more nucleotide excision repair genes in a biological sample from the patient, wherein the presence of one or more mutations in the one or more nucleotide excision repair genes indicates a sensitivity to the treatment by a platinum-based antineoplastic agent,
wherein the cancer is selected from the group consisting of bladder cancer, gastric cancer, prostate cancer, colorectal cancer, lung adenocarcinoma, cutaneous melanoma, head and neck squamous cell carcinoma, low-grade glioma, cervical cancer, ovarian cancer, renal cancer and breast cancer, and
wherein the one or more nucleotide excision repair genes comprise one or more genes elected from the group consisting of ERCC2, ERCC3 and ERCC5 genes.

2. The method of claim 1, wherein the cancer is bladder cancer.

3. The method of claim 2, wherein the bladder cancer is muscle-invasive urothelial carcinoma.

4. The method of claim 1, wherein the one or more nucleotide excision repair genes comprise ERCC2 gene.

5. The method of claim 4, wherein the one or more nucleotide excision repair genes comprise ERCC2 gene and one or more genes selected from the group consisting of ERCC1, ERCC3, ERCC4, ERCC5, ERCC6, ERCC8, CCNH, CDK7, CETN2, DDB1, DDB2, LIG1, MNAT1, MMS19, RAD23A, RAD23B, RPA1, RPA2, TFIIH, XAB2, XPA and XPC genes.

6. The method of claim 1, further comprising the step of determining in the biological sample the presence of somatic mutation in one or more additional genes selected from the group consisting of ATM, PARP1, ATRX, PMS1, BAP1, PMS2, BARD1, ERCC5, POLE, BLM, FANCA, RAD50; MLH1, RAD51, BRCA1, BRCA2, MRE11A, RAD51B, BRIP1, MSH2, RAD51C, CHEK1, MSH6, RAD51D, CHEK2, NBN, RAD52, FANCC, PALB2, BRIP1, FANCG, FANCD2, FANCF, FANCL, FANCI, FANCJ and FANCB genes.

7. The method of claim 1, wherein the platinum-based antineoplastic agent is cisplatin.

8. The method of claim 1, further comprising the step of administering an effective amount of the platinum-based antineoplastic agent into the patient if a somatic mutation is found in one of said one or more nucleotide excision repair genes.

9. A method of treating a patient suffering from cancer, the method comprising the steps of:
determining the presence of somatic mutation in one or more nucleotide excision repair genes in a biological sample from the patient; and
administering to the patient an effective amount of a platinum-based antineoplastic agent, if one or more mutations are found in the one or more nucleotide excision repair genes in the biological sample,
wherein the cancer is selected from the group consisting of bladder cancer, gastric cancer, prostate cancer, colorectal cancer, lung adenocarcinoma, cutaneous melanoma, head and neck squamous cell carcinoma, low-grade glioma, cervical cancer, ovarian cancer, renal cancer and breast cancer, and
wherein the one or more nucleotide excision repair genes comprise one or more genes elected from the group consisting of ERCC2, ERCC3 and ERCC5 genes.

10. The method of claim 9, wherein the cancer is bladder cancer.

11. The method of claim 10, wherein the bladder cancer is muscle-invasive urothelial carcinoma.

12. The method of claim 9, wherein the one or more nucleotide excision repair genes comprise ERCC2 gene.

13. The method of claim 12, wherein the one or more nucleotide excision repair genes comprise ERCC2 gene and one or more genes selected from the group consisting of ERCC1, ERCC3, ERCC4, ERCC5, ERCC6, ERCC8, CCNH, CDK7, CETN2, DDB1, DDB2, LIG1, MNAT1, MMS19, RAD23A, RAD23B, RPA1, RPA2, TFIIH, XAB2, XPA and XPC genes.

14. The method of claim 9, further comprising the step of determining in the biological sample the presence of somatic mutation in one or more additional genes selected from the group consisting of ATM, PARP1, ATRX, PMS1, BAP1, PMS2, BARD1, ERCC5, POLE, BLM, FANCA, RAD50; MLH1, RAD51, BRCA1, BRCA2, MRE11A, RAD51B, BRIP1, MSH2, RAD51C, CHEK1, MSH6, RAD51D, CHEK2, NBN, RAD52, FANCC, PALB2, BRIP1, FANCG, FANCD2, FANCF, FANCL, FANCI, FANCJ and FANCB genes.

15. The method of claim 9, wherein the platinum-based antineoplastic agent is cisplatin.

16. A kit for determining sensitivity to a platinum-based antineoplastic agent in a cancer patient, comprising: one or more synthetic oligonucleotides that specifically hybridizes to human ERCC2 gene; and
one or more reagents for processing a biological sample to obtain nucleotide molecules.

17. The kit of claim 16, further comprising one or more reagents for amplifying a portion of the human ERCC2 gene with the one or more synthetic oligonucleotides.

18. The kit of claim 16, reagents for DNA or RNA purification.

19. The kit of claim 16, wherein the cancer patient is suffering from a cancer selected from the group consisting of bladder cancer, gastric cancer, prostate cancer, colorectal cancer, lung adenocarcinoma, cutaneous melanoma, head and neck squamous cell carcinoma, low-grade glioma, cervical cancer, ovarian cancer, renal cancer and breast cancer.

20. The kit of claim 16, wherein the cancer patient is suffering from urothelial carcinoma and wherein the platinum-based antineoplastic agent is cisplatin.
